(19) United States
(12) Patent Application Publication
(10) Pub. No.: US 2004/0043475 A1
(43) Pub. Date: Mar. 4, 2004

(54) Exhaust gas purification system

(76) Inventors: Friedrich Proll, Eberschwang (AT); Manfred Lisberger, Riedau (AT)

Correspondence Address: MERCHANT & GOULD PC, P.O. BOX 2903, MINNEAPOLIS, MN, 55402-0903, US

(21) Appl. No.: 10/398,107

(22) PCT Filed: Oct. 2, 2001
(86) PCT No.: PCT/AT01/00310

(30) Foreign Application Priority Data
Oct. 3, 2000 (AT) A 1679/2000
Apr. 19, 2001 (AT) A 635/2001

(51) Int. Cl. A61L 9/01

(52) U.S. Cl. 435/266

(57) ABSTRACT

The invention relates to a system for purifying exhaust gases, comprising at least two filter stages (1, 2, 3), including at least one filter stage (1) for purifying the exhaust gases from particulate and aerosol-containing substances, and at least one filter stage (2) in the form of a biological filter for purifying the exhaust gases from organic compounds, aerosols and other odor-intensive substances by means of microorganisms. The second filter stage comprises a mass transfer zone (9) in which the exhaust gases are transformed from the gas phase to the liquid phase by the atomization of wash water. The aim of the invention is to provide a simple and inexpensive system with which high purification rates can be achieved. To this end, at least one filter stage (1, 3) is configured as a spray tower or wet electric filter using wash water, and the system is provided with means for maintaining a constant temperature in the thermophilic temperature range of from 45° C. to 75° C. in the filter stage (2) that is configured as a biological filter.

The invention relates to a system for purifying waste gases loaded with dust, aerosols and with volatile organic carbon compounds, in particular waste-gases from drying systems for biogenous crude materials, comprising at least two successively arranged filter stages including at least one filter stage for purifying the waste-gases from particulate and aerosol-containing ingredients, and at least one filter stage for purifying the waste gases from organic compounds, aerosols as well as further odor-intensive substances by means of microorganisms, which filter stage is designed as a biological filter, with a mass transfer zone in which the transfer of the waste gases from the gaseous phase into the liquid phase occurs by the atomization of wash water.

The term “waste gases” includes both gases derived from combustion processes and outgoing air flows which have not been preceded by a combustion.

Even though the described method and the system, respectively, is described particularly for the purification of waste gases from drying plants for organic natural substances, such as wood, loppings or compost, but also desiccating plants in the field of foodstuffs, such as cereal drying or the production of animal feed, also other applications are conceivable.

With such systems, depending on the respective material being dried and the mode of drying, organic dust particles, gaseous organic and partly very odor-intensive carbon compounds (VOC: volatile organic carbon), water vapor, inorganic flue gas components and flue ash are discharged. What is specific of these out-going air flows is their high water vapor dew point of from 45° C. to 75° C., which is a consequence of the high degree of humidity of the outgoing air.

To separate the above-mentioned impurities, dry-operating purification systems, such as cyclones, fibrous or stratified bed filters, and dry electric filters are known. However, these systems are insufficient for separating fine dusts and organic components.

To separate the finest dust particles, smallest liquid droplets and particles and gaseous organic components, wet-operating systems have been developed. Thus, EP 358 006 B1 describes a method and a system for purifying waste gases derived from drying plants, which system consists, e.g. of a dry separator designed as a cyclone, followed by a washing and condensing installation, for a pre-separation. Subsequently, the waste gas flows through an electric filter. Since this system has to be switched off during purification, large amounts of crude gas may escape unpurified into the atmosphere during this time. Moreover, these systems generally operate in a higher temperature range of up to 75° C., in which the separating performance in terms of gaseous organic compounds is limited with about 20%. By this, only slight degrees of odor reduction are attainable.

A further development of this method is described in EP 740 963 A1, wherein by a multi-stage arrangement of the system, a continuous operation is also ensured during the cleaning cycles. To enhance the cleaning effect, at least one of the filter stages of the electric filter is designed as an air-cooled tube system. As a consequence, condensation water continually forms on parts of the electric filter and rinses off a large portion of the dusts and of the organic substances already during operation. A further inventive feature of this system is to be seen in the optic impression of the escaping pure gas flow. While conventional systems exhibit a significant waste gas plume at a waste gas temperature of around 65° C. (due to the contents of water vapour), these plants do not exhibit an optically conspicuous cloud of fume, since pre-heated, unsaturated ambient air is admixed to the purified waste gas flow before it enters the vent stack.

Very high degrees of effectiveness as regards dust and organic carbon compounds are attained by thermal afterburning systems which, however, cause enormous operating costs.

In EP 960 648 A1, a method is described in which in a first step for separating solids, the waste gas is pre-washed with a first wash water circulation, and in a second step is purified from organic components in an activated sludge treatment as second wash water circulation. At the heart of this combination of methods is a lowering of temperature after the first step to below 50° C. By this, a relatively simple handling of the biological degradation process is provided, since the microorganisms in the mesophilic temperature range (up to about 45° C.) are not sensitive to temperature fluctuations. Disadvantages of this method are the high investment costs and operating costs, since either very large amounts of cooling air and, thus, a considerable enlargement of the plant is required, or additional costs of the plant are incurred by the required heat exchanger and cooling towers.

EP 0 111 302 B1 describes a method for biofiltering, in which a biofilter is used which commonly consists of organic carrier material, such as bark mulch, or the like, and is degraded simultaneously with the biological purification. As a consequence, the material is used up and no longer available for cleaning after a certain period.

Also DE 31 18 455 A1 describes a method for the wet purification of organically contaminated hot outgoing air from enamelling lines in which the outgoing air to be purified is cooled, and the condensate droplets formed are separated from the outgoing air. A suitable temperature range of from 10 to 35° C. is preferred for mesophilic microorganisms. Also in this instance a biofilter is used, as is in the last-mentioned document.

Also RU 2 106 184 C shows a two-stage waste gas purification process which uses a biofilter in the mesophilic temperature range.

The present invention has as its object to provide a system of the above-indicated type, which is simpler and less expensive to produce than conventional systems, and with which high purification rates are attainable. Disadvantages of known plants are to be reduced or avoided, respectively.

The object of the invention is achieved in that at least one filter stage is designed as a spray scrubber or wet electric filter by using wash water, and that means for maintaining a constant temperature level in the thermophilic temperature range of from 45° C. to 75° C. are provided in the filter stage that is configured as a biological filter. The degradation of pollutants occurs such that in a filter stage for separating dusty ingredients as well as aerosols, inorganic flue gas components as well as organic finest particles, such as resins, a pre-separating unit, e.g. in the form of a wet electric filter or a spray scrubber, is provided, and the waste gas is purified from the organic carbon components in a further filter stage. The biological purification is effected by so-called thermophilic microorganisms in the temperature range of between 45° C. and 75° C. The microorganism population takes up the pollutants in their metabolism. Subsequently, the pollutants are degraded to harmless compounds, such as carbon dioxide and water vapor, by oxygen consumption. A portion of the pollutants is also used to multiply the microorganisms, which entails an increase of the microorganisms present in the system, also called bio-mass. The degradation process proper occurs in a temperature range of between 45° C. and 75° C., where only certain, so-called thermophilic, microorganisms (e.g. Bacillus stearothermophilus, Thermoactinomyces vulgaris) have their growth maximum. The purification stages can be arranged, e.g., as follows, depending on the specific requirements:

Variant 1: spray scrubber—bioactive scrubber—wet electric filter

Variant 2: spray scrubber—wet electric filter—bioactive scrubber

Variant 3: spray scrubber—wet electric filter

Variant 4: spray scrubber—bioactive scrubber

Variant 5: bioactive scrubber—wet electric filter

Variant 6: wet electric filter—bioactive scrubber.

The thermophilic temperature range is very demanding for the degradation process proper. To balance out temperature peaks during operation of the biological filter stage, to keep the operating temperature of the subsequent filter stage in the optimum temperature range, and to merely allow for a slight fluctuation range in the operating temperature of e.g. +/−2° C., according to the invention the temperature is regulated in the filter stage that is designed as a biological filter. Depending on the specific requirements, the filter stages may be arranged as spray scrubbers or wet electric filters and in any arrangement desired, i.e. upstream or downstream of the filter stage that is designed as biological filter.

The filter stages contain reservoirs for the wash water with which the impurities are extracted from the waste gases. Usually, the wash water is introduced from the reservoir into the separating unit by means of a pump and returned into the reservoir in circulation together with the impurities of the waste gas. From the reservoir, the impurities are separately discharged.

According to a further characteristic of the invention, a common reservoir is provided for at least two filter stages.

The means for maintaining the constant temperature level may be formed by at least one, preferably air-filter-provided, air supply duct for supplying ambient air in at least one filter stage. Such an air cooling which may be before or directly into the respective filter stage, is very cost-effective and simple to provide.

In addition thereto or alternatively, the means for maintaining the constant temperature level can also be formed by at least one heat exchanger in a water circulation of at least one filter stage. Yet, it is also possible to incorporate a heat exchanger directly into the waste gas connecting duct in at least one filter stage.

According to a further feature of the invention, nozzles or the like are provided in the mass transfer zone of the filter stage that is designed as a biological filter, for atomizing wash water in the waste gas flow. Likewise, fixed installations in the form of so-called scrubber bottoms or the like, may be provided to attain a high mass transfer, or the waste-gas is guided over so-called tower packings, drippers or the like, for the fine distribution of the gas and water phases, and is sprinkled with the circulating liquid phase, also called wash water. In the mass transfer zone, the pollutants of the outgoing air transfer into the liquid phase.

Advantageously, the microorganisms in the filter stage designed as the biological filter are suspended in the wash water and/or immobilized on installations in the mass transfer zone, such as tower packings, drippers or the like. The microorganisms take up the pollutants dissolved in the wash liquid. With this, a regeneration, or purification, respectively, of the wash water occurs, which then again can take up pollutants from the outgoing air. In terms of time, the regeneration occurs partially already in the mass transfer zone and partially in the reservoir for the wash water.

The reservoir of the filter stage that is designed as a biological filter may be designed as a conventional activated sludge basin, or, for higher specific degradation capacities, it may also be equipped with inert tower packings or immersion bodies on which those microorganisms will adhere which are responsible for the degradation of the pollutants taken up from the outgoing air. Proportionally, a part of the microorganisms may also be immobilized on the installations of the mass transfer zone and responsible for the degradation of the pollutants.

To supply the microorganisms in the reservoir with oxygen or nutrients, ducts which optionally comprise valves or the like are provided for the metered supply of oxygen and nutrients. In addition, a duct for an optional additional metering of organically loaded waste water may be provided so as to supply the microorganisms with a load of impurities in facility downtimes.

To further increase the efficiency, it may be provided for the wash water used in a filter stage to be biologically purified. For this purpose, a connecting duct is arranged between the reservoirs of two filtering stages.

According to a further feature of the invention, it is provided for the arrangement of a circulation between the mass transfer zone of the filter stage that is provided as a biological filter, and the reservoir of that filter stage, in which a defined amount of liquid is continually circulated.

To keep the waste water amounts on the lowest level, yet still allow for a removal of the forming biomass and/or solids from the system, a solids separator is provided on the at least one reservoir. This solids separator may be a common secondary settler, as in conventional sewage treatment plants. Yet also a discharge of activated sludge may be effected by installations, such as baffle plate thickeners or by the installation of a decanter. If due to high shearing forces a reduced settling behavior of the activated sludge is observable, an addition of flocculents may be a remedy.

If a water treatment plant is arranged to follow the solids separator, the dissolved inorganic ingredients can be removed. This water treatment unit may, e.g., be realized in the form of a reverse osmosis.

According to a further feature of the invention it is provided that at the start-up of the purification plant as well as if the temperature is fallen below, the waste gases are guided past at least one filter stage for stabilizing the temperature. By such a measure, which preferably is realized by a bypass with appropriate valves arranged in parallel to the filter stage, e.g. at the start-up or in the standby operation of the production system with the low waste gas temepratures involved, a temperature lowering in the biological unit can be avoided.

To protect in particular the filter stage that is designed as a biological filter, from a lowering of the temperature, in particular during start-up of the purification plant as well as when the waste-gas temperature is fallen below, the bypass is arranged in parallel to the mass transfer zone of the filter stage that is designed as a biological filter.

Before the waste gases that have been purified from pollutants are delivered to the atmosphere, liquid droplets may be separated from the waste gases, e.g. via a droplet separator. In such a droplet separator, the liquid droplets of waste-gas which after the mass transfer is the humid and saturated are retained. To rinse the droplet separator from solids, the former is provided with a cleaning system, either fresh water or wash water being usable as said cleaning agent.

Besides the temperature regulation, also monitoring of the important waste-water technical parameters, such as pH, electric conductivity, oxygen content, chemical oxygen demand (COD), biological oxygen demand (BOD, BOD5), activated sludge content as well as the contents of phosphate, nitrate, nitrite, ammonium, as well as extremely toxic poisons, such as formaldehyde, which is contained in many waste gases from dryers is of importance. Monitoring with appropriate measuring devices may be at certain points of time or continuous. To be able to keep the microorganisms alive in sufficient numbers, is is provided that the latter are supplied with oxygen and nutrients. To maintain a minimum content of dissolved oxygen in the wash water reservoir, the latter preferably is provided with a continuous gas supply. This may be effected, e.g., by immersion aerators or diaphragm-disk aerators.

According to a further feature of the invention it is provided that the atomization of the wash water in the filter stage designed as a spray scrubber is effected by injection via one or several nozzle levels arranged in co-current with and/or counter-current to the air flow which flows in a horizontally or vertically arranged air channel.

The invention will be explained in more detail by way of the enclosed Figures which show block diagrams of different variants of the present invention. Therein,

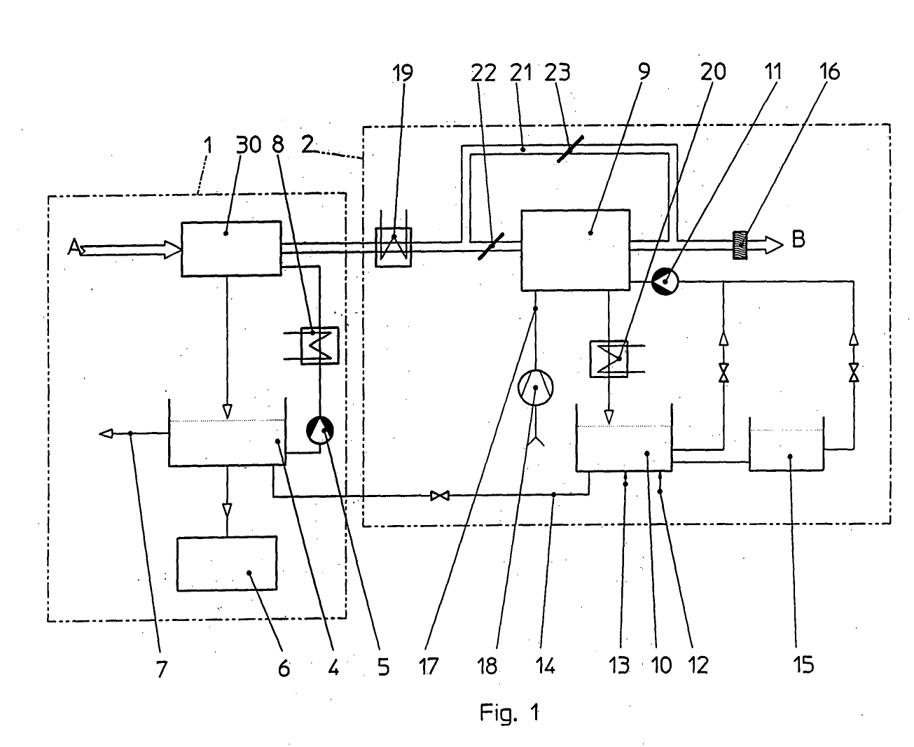

FIG. 1 shows a block diagram of an embodiment variant of a double-stage waste gas purification system,

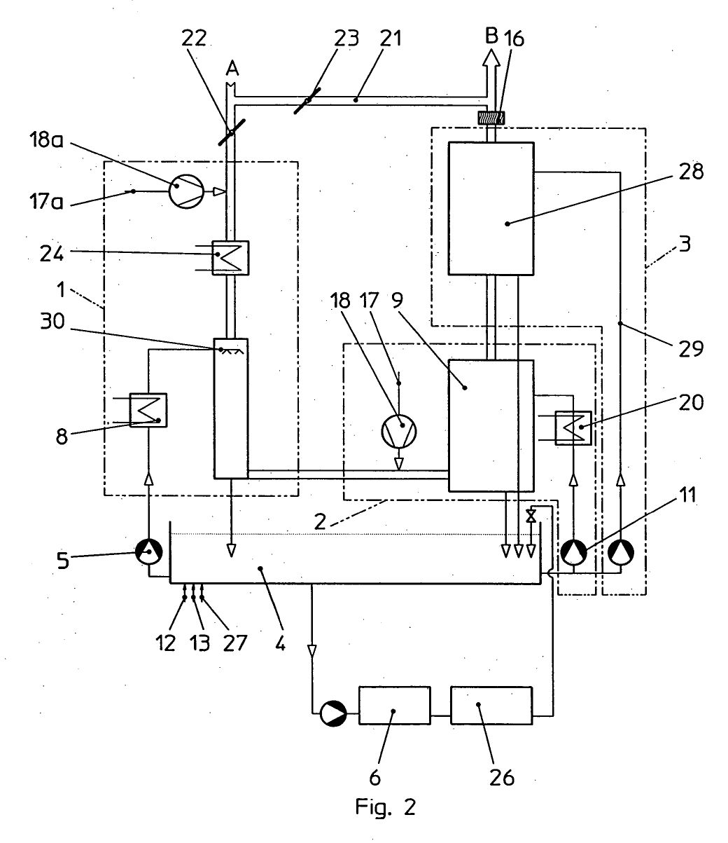

FIG. 2 shows a variant of a triple-stage purification system specifically suitable for the treatment of waste gas flows including gaseous organic pollutant components, dust, as well as aerosols, and

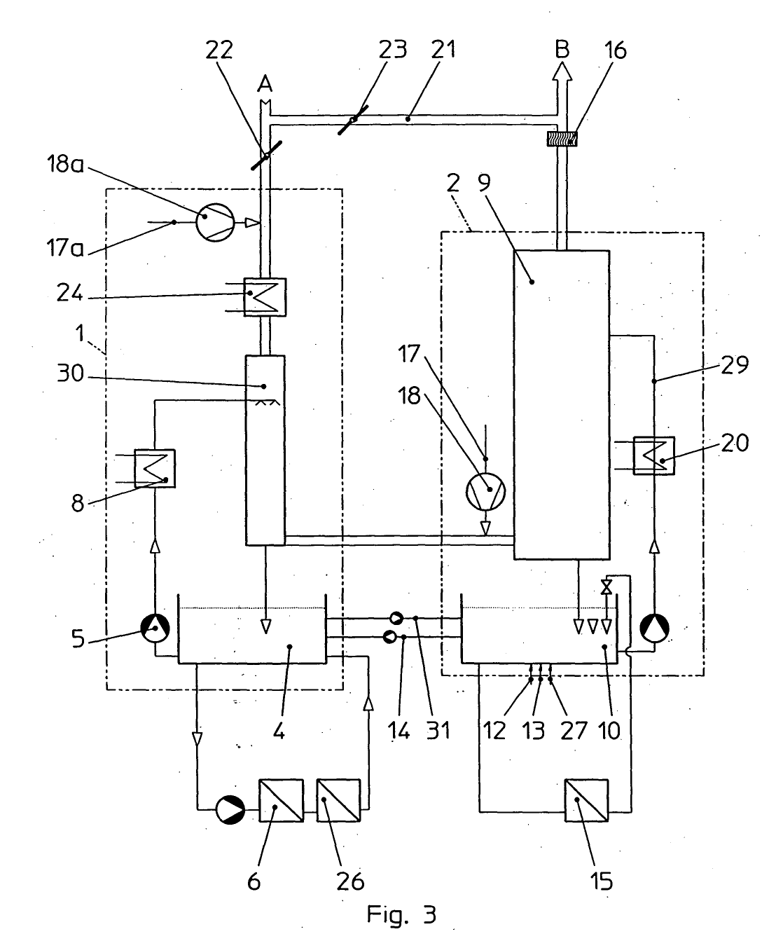

FIG. 3 shows a variant of a double-stage purification system which removes gaseous inorganic components from the outgoing air in addition to the aforementioned impurities.

According to FIG. 1, crude gas A arriving, e.g., from a wood chip drying system enters a first filter stage which serves to separate the dust particles and aerosols as well as inorganic ingredients. Accordingly, the crude gas A enters a pre-separating unit 30 designed, e.g., as a wet electric filter. Such filters are particularly suitable for separating dusty and areosol-containing pollutants and inorganic pollutants, such as, e.g., HCl and SO₂. For this purpose, water is introduced from a water container 4 into the pre-separating unit 30 by means of a pump 5, and recirculated with the impurities into the water container 4. From this water container 4, the solids are removed via a solids separator 6. The waste water is disposed of via a duct 7. The crude gas which, e.g., is derived from the wood chip drying system, because of its high moisture content has a high dew point, e.g. in the range of from 45° C. to 75° C. Therefore, it may be necessary for optimum functioning of the biological filter stage 2, especially for levelling temperature peaks, to cool the circulated purification water, e.g. by means of a heat exchanger 8.

The crude gas which has been freed from solid impurities then reaches the second filter stage 2, in which the crude gas mainly is freed from organic pollutants. The second filter stage 2 at first has a mass transfer zone 9 in which the mass transfer of the gaseous ingredients occurs from the waste gas to the circulating liquid phase. Such a mass transfer zone 9 can be realized by the atomization of water into the air flow of the crude gas, or by installations, such as tower packings, drippers or scrubber bottoms, in a conventionally known manner. The wash water is guided in circulation via a reservoir 10 and via appropriate ducts by means of a pump 11 via the mass transfer zone 9. The reservoir 10 may have the form of a conventional activating basin or with integrated immersion bodies. The degradation of the pollutants taken up in the mass transfer zone 9 is effected by microorganisms. These microorganisms may be present suspended in wash water (so-called activated sludge) and/or they may deposit on the installations in the mass transfer zone (e.g. on the tower packets or drippers). If so-called immersion bodies are present in the wash water, the microorganisms can deposit also on these bodies accordingly. According to the invention, the biological purification is carried out in the thermophilic temperature range, i.e. in the range of from 45° C. to 75° C. In this temperature range, certain thermophilic microorganisms exhibit their optimum growth. In order to keep the temperature fluctuation range limited, the second filter stage 2 is supplied with ambient air via duct 17, optionally via a fan 18. In the supply duct 17, optionally an air filter (not shown) may be provided. Further possible ways of regulating the temperature as well as of levelling temperature peaks are the installation of a heat exchanger 20 in the wash water circulation of the second filter stage 2 or a heat exchanger 19 in the waste gas connection duct between the first filter stage 1 and the second filter stage 2.

For the purpose of an optimal temperature regulation, parameters, such as the pH, conductivity, oxygen content, the chemical oxygen demand COD (that oxygen which is required for a complete oxidation), furthermore, the biological oxygen demand BOD (determination of the content of microbially degradable organic material via the amount of oxygen required for eliminating the organic material by breathing, by means of microorganisms) as well as activated sludge content and content of phosphate, nitrate, nitrite and ammonium. Moreover, the determination of the content of toxic poisons, such as formaldehyde, in the wash water is suitable.

To promote the growth of the microorganisms, the reservoir 10 is supplied with oxygen and nutrients via ducts 12, 13. The reservoir 10 may also be designed such that it also allows a possible degradation of organically loaded waste waters in addition to the biological degradation of the outgoing air ingredients. To this end, a connection is formed via a duct 14 between the wash water circulation of the pre-separator 30 as the first filter stage 1, and the wash water circulation of the biological purification unit as the second filter stage 2. Likewise, a further duct (not illustrated) to the reservoir 10 may be provided for the treatment of further waste water flows within the operating system. Thus, e.g., in facility downtimes, in which no outgoing air is incurred and therefore, no outgoing air ingredients are present, the microorganisms may be supplied with nutrition by means of the waste water load introduced, and a dying of the biomass may be avoided. This is especially important when the system causing the waste gases is put into operation again, since in this manner a high degradation activity of the microorganisms will be reached very quickly and, consequently, a high purification performance can be registered. The pollutants taken up into the metabolism of the microorganisms can be degraded to safe compounds, such as carbon dioxide and water vapor, under oxygen consumption, and used proportionally for the growth of the microorganisms. Excess of biomass may, if required, be discharged by a solids separating system 15, e.g. in the form of a secondary settler, baffle plate thickener or decanter. The liquid medium guided via the soldis separating system 15 is returned into the circulation.

To avoid a drop in the operating temperature of the purifying system in case of a short-term or longer-term reduction of the dew point temperature on account of interruptions of the production, according to a further feature of the invention, a bypass 21 is provided which guides the waste-gas around the mass transfer zone 9. for this purpose, respective valves 22 and 23 are provided in the bypass 21 and in the duct into the mass transfer zone 9, which valves will be activated upon demand. If the gas is guided around the mass transfer zone 9, i.a. a cooling of the biological filter stage and, thus, an inhibition of the microbiological activity of the thermophilic microorganisms will be prevented. As has already been mentioned, the mass transfer zone 9 may be realized by simple spray scrubbers finely spraying the wash water so that the pollutants of the gas will transit into the water and can be fed to the microorganisms present in the water. Moreover, scrubber bottoms where wash water is sprayed via small apertures in the scrubber bottoms can be used. Also so-called tower packings or drippers, respectively, these are structural bodies that are sprinkled with water, can be employed. Via the sprinkling, also nutrients can be delivered for the microorganisms immobilized on the installations of the mass transfer zone.

Before being discharged to the atmosphere as pure gas B, the gas which has been purified from the organic pollutants will be guided over a droplet separator 16 so as to remove the finest liquid droplets.

FIG. 2 shows a triple-stage filter arrangement in which the crude gas A reaches a first filter stage 1 which is designed as a spray scrubber and serves to separate dust particles and organic ingredients. For this purpose, wash water from the reservoir 4 is atomized in the spraying zone of the pre-separating unit 30 with the assistance of a pump 5, whereby the waste gas ingredients at first become absorbed. The atomization is effected by injection via one or several nozzle level(s) arranged in co-current with and/or counter-current to the air flow, which flows in a horizontally or vertically arranged air channel. To level temperature peaks, the purifying water guided in circulation may be cooled, e.g. by a heat exchanger 8.

Further possible means for a temperature regulation as well as for equalizing temperature peaks are the installation of a heat exchanger 24 into the crude gas supply duct to the pre-separator 30 or the supply of ambient air via a duct 17a and an auxiliary fan 18a into the crude gas feed duct.

The reservoir 4 may have the shape of a conventional activation basin or it may be designed to have integrated immersion bodies. The degradation of the organic pollutants taken up in the first filter stage 1 is mainly effected in the reservoir 4 via a biological purification, with the biomass increasing as the operation continues. Moreover, also dusty particles will increase which are removed from the waste gas in the pre-separating unit 30. To remove biomass and dust particles, a solids separator 6, e.g. in the form of a secondary settler, a baffle plate thickener or a decanter, are installed. If needed, this system may be followed by a liquid/liquid water treatment unit 26, i.e. in the form or a reverse osmosis.

To promote the growth of the microorganisms, the reservoir 4 will be supplied with oxygen and nutrients via ducts 12, 13. The reservoir 4 may also be designed such that it will also allow for a possible degradation of organically loaded waste water via duct 27, in addition to the biological degradation of the outgoing-air ingredients.

The degradation of the pollutants taken up in the mass transfer zone 9 of the second filter stage 2 formed as a biological filter is effected by microorganisms.

To separate the finest dusts and aerosols, according to the invention a third filter stage 3 designed as a wet electric filter 28 is provided as a further system component, which third filter stage will be rinsed at periodic intervals, water from the common reservoir 4 being directly used as rinsing water via duct 29.

Here, too, a bypass 21 may be provided between the duct for the crude gas A and the duct for the crude gas B, which bypass will guide the waste gas around the purification system. For this purpose, the valves 22 and 23 are provided which will be activated upon demand.

FIG. 3 shows a further double-stage arrangement, in which the crude gas A is guided into the first filter stage 1, designed as a pre-separating unit 30, such as, e.g., a wet electric filter or spray scrubber. For this purpose, water from the reservoir 4 of the first filter stage 1 is atomized into the pre-separating unit 30, whereby the waste gas ingredients at first will be absorbed. As the operation continues, solids and absorbed inorganic waste-gas ingredients will increase in the reservoir 4. To discharge these substances, a solids separator 6 is installed. To remove the dissolved inorganic ingredients, this system may be followed by a liquid/liquid water treatment unit 26, e.g. in the form of a reverse osmosis.

The crude gas which has been freed from solid pollutants and from gaseous inorganic ingredients now will get into the second filter stage 2 designed as a biological filter; the latter at first includes a mass transfer zone 9 in which the mass transfer of the gaseous organic ingredients from the waste gas to the circulating liquid phase occurs. Such a mass transfer zone 9 can be realized in a common conventional manner by atomizing water into the air flow of the crude gas, or via installations, such as tower packings, drippers or scrubber bottoms. The degradation of the pollutants taken up in the mass transfer zone is effected by microorganisms. These microorganisms may be present suspended in wash water (so-called activated sludge) and/or they may adhere to the installations of the mass transfer zone (e.g. on the tower packings or drippers). If so-called immersion bodies are present in the wash water, the microorganisms may sediment also on these bodies accordingly. To keep the temperature-fluctuation range limited, also in this stage heat exchangers 20 may be installed, or fresh air may be supplied, respectively, via a duct 17 and an auxiliary fan 18.

The reservoir 10 of the biological stage may in turn be designed as a conventional activating basin or with integrated immersion bodies. To discharge the biomass, a separate solids separator 15 may be installed. However, also a partial stream 6 may be supplied from the reservoir 10 via duct 14 to the solids separator 6 of the first filter stage 1. Likewise, via a further duct 31 a connection from the reservoir 4 of the first filter stage 1 to the reservoir 10 of the second filter stage 2 may be arranged.

To promote the growth of the microorganisms, the reservoir 10 will be supplied with oxygen and nutrients via ducts 12, 13. The reservoir 10 may also be designed such that it allows for a possible degradation of organically loaded waste waters via duct 27 as well as via duct 29, and also for a biological treatment of the wash water of the first filtering system 1, in addition to the biological degradation of the outgoing-air ingredients.

In order to avoid a drop of the operating temperature of the purification system in case of a short-term or longer reduction of the dew point temperature by interruptions of the production, a bypass 21 may be provided which can be activated via valves 22 and 23 upon demand.

With the method according to the invention, optimum purification conditions can be provided. Moreover, more cost-effective and simpler purification systems may be used, since no or only little cooling of the waste gases is required and thus the usually enormous amounts of cooling water and enormously complex cooling towers are avoided.

1. A system for purifying waste gases loaded with dust, aerosols and with volatile organic carbon compounds, in particular waste-gases from drying systems for biogenic crude materials, comprising at least two successively arranged filter stages (1, 2, 3) including at least one filter stage (1) designed as a spray scrubber or wet electric filter using wash water, for purifying the waste-gases from particulate and aerosol-containing ingredients, and at least one filter stage (2) for purifying the waste gases from organic compounds, aerosols as well as further odor-intensive substances by means of microorganisms, which filter stage (2) is designed as a biological filter, with a mass transfer zone (9) in which the transfer of the waste gases from the gaseous phase into the liquid phase occurs by the atomization of wash water, characterized in that means for maintaining a constant temperature level in the thermophilic temperature range of from 45° C. to 75° C. are provided in the filter stage (2) that is designed as a biological filter, and that furthermore installations for immobilizing the microorganisms are provided in the mass transfer zone (9).

2. A system according to claim 1, characterized in that the filter stages (1, 2, 3) comprise reservoirs (4, 10) for the wash water.

3. A system according to claim 2, characterized in that a common reservoir (4) is provided for at least two filter stages (1, 2, 3).

4. A system according to any one of claims 1 to 3, characterized in that the means for maintaining the constant temperature level are formed by at least one, preferably air-filter-provided, air supply duct (17, 17a) for supplying ambient air in at least one filter stage (1, 2, 3).

5. A system according to any one of claims 1 to 4, characterized in that the means for maintaining the constant temperature level are formed by at least one heat exchanger (8, 20) in a water circulation of at least one filter stage (1, 2, 3).

6. A system according to any one of claims 1 to 5, characterized in that the means for maintaining the constant temperature level are formed by at least one heat exchanger (19, 24) in a duct of the waste gases in at least one filter stage (1, 2, 3).

7. A system according to any one of claims 1 to 6, characterized in that in the mass transfer zone (9) of the filter stage (2) that is designed as a biological filter, nozzles or the like are provided for atomizing wash water in the waste gas flow, scrubber bottoms or the like are provided for the fine distribution of the gas and water flow, or tower packings, drippers or the like are provided for the fine distribution of the gas and water phases.

8. A system according to any one of claims 1 to 7, characterized in that the installations in the mass transfer zone (9) are formed by tower packings, drippers or the like.

9. A system according to any one of claims 1 to 8, characterized in that the reservoir of the filter stage (2) designed as a biological filter is designed as an activated sludge basin.

10. A system according to claim 9, characterized in that the reservoir (4, 10) includes tower packings or immersion bodies.

11. A system according to claim 9 or 10, characterized in that the reservoir (4, 10) comprises ducts (12, 13, 14) for the metered supply of oxygen, nutrients or organically loaded waste waters.

12. A system according to any one of claims 1 to 11, characterized in that a connecting duct (14) is arranged between the reservoirs (4, 10) of two filtering stages (1, 2, 3).

13. A system according to any one of claims 1 to 12, characterized in that a circulation is arranged between the mass transfer zone (9) of the filter stage (2) that is designed as a biological filter, and the reservoir (10) of that filter stage (2).

14. A system according to any one of claims 1 to 13, characterized in that a solids separator (6, 15) is provided on the at least one reservoir (4, 10) for discharging an excess of biomass and/or solids.

15. A system according to claim 14, characterized in that a water treatment unit (26) is arranged to follow the solids separator (6, 15).

16. A system according to any one of claims 1 to 15, characterized in that a bypass (21) comprising at least one valve (23) is provided in parallel to at least one filter stage (1, 2, 3).

17. A system according to claim 16, characterized in that the bypass (21) is arranged in parallel to the mass transfer zone (9) of the filter stage (2) that is designed as a biological filter.

18. A system according to any one of claims 1 to 17, characterized in that at least one droplet separator (16) is arranged in the duct for the purified waste-gases for separating finest liquid droplets.

19. A system according to any one of claims 1 to 18, characterized in that measuring devices for parameters such as pH, electric conductivity, oxygen content, chemical oxygen demand, biological oxygen demand, activated sludge content as well as the contents of phosphate, nitrate, nitrite, ammonium, formaldehyde etc are arranged in the washing circulation of at least one filter stage (1, 2, 3).

20. A system according to any one of claims 1 to 19, characterized in that the atomization of the wash water in the filter stage (1) designed as a spray scrubber is effected by injection via one or several nozzle levels arranged in co-current with and/or counter-current to the air flow which flows in a horizontally or vertically arranged air channel.
